(19) United States
(12) Patent Application Publication
(10) Pub. No.: US 2018/0235983 A1
(43) Pub. Date: Aug. 23, 2018

(54) METHOD OF PREPARING NANOPARTICULATE TOPICAL COMPOSITION

(71) Applicant: SUN PHARMA ADVANCED RESEARCH COMPANY LTD., Mumbai (IN)

(72) Inventors: Madhusudhan BOMMAGANI, Baroda (IN); Subhas Balaram BHOWMICK, Baroda (IN); Prashant KANE, Baroda (IN); Vaibhav DUBEY, Baroda (IN)

(73) Assignee: SUN PHARMA ADVANCED RESEARCH COMPANY LTD., Mumbai (IN)

(21) Appl. No.: 15/552,887

(22) PCT Filed: Feb. 25, 2016
(86) PCT No.: PCT/IN2016/050067

§ 371 Date: Aug. 23, 2017

(30) Foreign Application Priority Data
Feb. 25, 2015 (IN) 627/MUM/2015

(51) Int. Cl. A61K 31/65; A61K 9/00; A61K 9/14

(52) U.S. Cl. CPC A61K 31/65; A61K 9/146; A61K 9/143; A61K 9/0014

(57) ABSTRACT

The present invention relates to a method of preparing a nanoparticulate topical composition of a water soluble, water-susceptible active ingredient or its pharmaceutically acceptable salt, the method comprising steps of milling the water soluble, water-susceptible active ingredient or its salt, a wetting agent and a non-aqueous liquid vehicle to obtain a non-aqueous nanosuspension and converting the non-aqueous nanosuspension into a topical composition.

### FIELD OF THE INVENTION

The present invention relates to a method of preparing a nanoparticulate topical composition of a water soluble, water-susceptible active ingredient or its pharmaceutically acceptable salt, the method comprising steps of milling the water soluble, water-susceptible active ingredient or its salt, a wetting agent and a non-aqueous liquid vehicle to obtain a non-aqueous nanosuspension and converting the non-aqueous nanosuspension into a topical composition.

### BACKGROUND OF THE INVENTION

Water soluble active ingredients that are susceptible to hydrolysis in the presence of water, are difficult to formulate. A major challenge in the development of topical compositions of these water soluble and water susceptible active drug lies in maintaining their physico-chemical stability. This is because such drugs are unstable in solution form and are sensitive to water, moisture and protic solvents. Further, the oxidative processes are also responsible for destabilizing many of these active agents in formulations leading to physico-chemical instability.

Since the drugs are susceptible to hydrolysis, compositions comprising aqueous phase or polar solvent are not feasible due to drug instability. Further, in case of non-aqueous dosage forms, the bio-availability of the water soluble and water-susceptible active ingredients becomes a major concern in that the composition do not show proper therapeutic effect upon topical application, due to lack of absorption or penetration.

There remains a medical need for a stable, commercially feasible, easy to manufacture and easy to use topical composition of a water soluble, water-susceptible active drug which on one hand is physically and chemically stable for the duration of its shelf life and on the other hand show optimum drug bio-availability and efficacy upon topical application. The present invention fulfills this need. The present inventors have surprisingly found a solution to the aforesaid problems by providing a method of preparing a nanoparticulate topical composition of water soluble, water-susceptible active ingredient which is physically and chemically stable. It was surprisingly observed that in the nanoparticulate topical composition developed by the present inventors, the active ingredient remained stable inspite of undergoing nanosizing, which otherwise results in formation of impurities.

### SUMMARY OF THE INVENTION

The present invention provides a method of preparing a nanoparticulate topical composition, said method comprising steps of—
i. mixing a water soluble, water-susceptible active ingredient or its pharmaceutically acceptable salt, one or more wetting agents, a non-aqueous liquid vehicle
ii. incorporating at least one inert grinding medium into the mixture of step (i),
iii. milling the mixture of step (ii),
iv. separating the at least one inert grinding medium from the milled mixture of step (iii) to obtain a non-aqueous nanosuspension, and
v. converting the non-aqueous nanosuspension of step (iv) into a topical composition wherein the method does not involve use of water.

### DESCRIPTION OF THE INVENTION

The term “nanoparticle” or “nanoparticulate” as used herein refers to the solid particles of active ingredient having a particle size in nanometer (nm), such that 90% of the particles (D₉₀) have a size less than 1000 nanometers (nms), i.e. D₉₀ is less than 1000 nanometers (nm). The solid particles consist of the active ingredient in that the solid particles are devoid of any other excipient which may either encapsulate the active ingredient, or embed the active ingredient within itself for example liposomally entrapped particle, or active ingredients entrapped in a porous structure of an excipient such as calcium or silica or any polymeric particles. It may be noted that the solid particles may include excipients adsorbed onto its surface, such as for example wetting agents, surfactants or surface stabilizers, which excipients are only adsorbed onto the surface of the active ingredient and there is no composite particle formed thereof.

The term “nanoparticulate composition” as used herein refers to compositions comprising the solid particles of active ingredient having particle size in nanometers, such that 90% of the particles have a size less than 1000 nm, i.e. D₉₀ is less than 1000 nms.

The particle size is expressed in terms of particle size distribution including values of D₉₀, D₅₀ and D₁₀, as measured by techniques such as laser light diffraction technique, photon correlation spectroscopy; sedimentation field flow fractionation, or disk centrifugation.

The phrase D₉₀ of less than Y nm—as used herein means that particle size distribution is such that at least 90% of the particles have a size/diameter of less than Y nm when measured by conventional techniques, such as laser light diffraction technique, photon correlation spectroscopy; sedimentation field flow fractionation, or disk centrifugation.

The phrase D₅₀ of less than X nm—as used herein means that particle size distribution is such that at least 50% of the particles have a size/diameter of less than X nm when measured by conventional techniques, such as laser light diffraction technique, photon correlation spectroscopy; sedimentation field flow fractionation, or disk centrifugation.

The phrase D₁₀ of less than Z nm—as used herein means that particle size distribution is such that at least 10% of the particles have a size/diameter of less than Z nm when measured by conventional techniques.

The term “non-aqueous” as used herein means free of added water. The topical nanoparticulate compositionS obtained according to the method of the present invention contains a liquid vehicle that is free of water. The term “liquid vehicle” as used herein includes a vehicle that can be poured from one container to another container or a vehicle can be sprayed or can form foam or any semisolid vehicle that can be squeezed out from a flexible container such as an ointments tube. In preferred embodiments, it includes a topical vehicle comprising pharmaceutically acceptable excipients employed in formulating topical dosage forms such as a gel, foam, an ointment, a suspension, an aerosol, a spray, a cream, a lotion.

The term “water soluble active ingredient” as used herein refers to therapeutically active drug substances that have a solubility greater then 1 mg per ml in water. The term “water-susceptible” as used herein refers to water soluble active ingredient that chemically degrades in the presence of water, either instantaneously or at a rate such that it does not remain within its specifications such as those specified as per ICH guidelines, over a shelf life period of up to 1 year. The term “water soluble, water-susceptible active ingredient” as used herein refers to therapeutically active drug substances that have a solubility greater than 1 mg per ml in water and that typically chemically degrades in the presence of water instantaneously or at a rate such that it does not remain within its specifications over a shelf life period of up to 1 year.

According to one embodiment of the present invention, there is provided a method of preparing a nanoparticulate topical composition, said method comprising steps of—
i. mixing a water soluble active ingredient or its pharmaceutically acceptable salt, one or more wetting agents, a non-aqueous liquid vehicle
ii. incorporating at least one inert grinding medium into the mixture of step (i),
iii. milling the mixture of step (ii),
iv. separating the at least one inert grinding medium from the milled mixture of step (iii) to obtain a non-aqueous nanosuspension, and
v. converting the non-aqueous nanosuspension of step (iv) into a topical composition wherein the method does not involve use of water.

According to another embodiment of the present invention, there is provided a method of preparing a non-aqueous nanosuspension of a water soluble active ingredient, said method comprising steps of—
i. mixing a water soluble active ingredient or its pharmaceutically acceptable salt, one or more wetting agents, a non-aqueous liquid vehicle
ii. incorporating at least one inert grinding medium into the mixture of step (i),
iii. milling the mixture of step (ii),
iv. separating the at least one inert grinding medium from the milled mixture of step (iii) to obtain a non-aqueous nanosuspension.

In this embodiment, the present invention provides a non-aqueous nanosuspension comprising the water soluble, water-susceptible active ingredient or its pharmaceutically acceptable salt, one or more wetting agents and a non-aqueous liquid vehicle, prepared according to the method hereinabove described.

According to one embodiment of the present invention, there is provided a nanoparticulate topical composition comprising nanoparticles of a water soluble, water-susceptible active ingredient or its pharmaceutically acceptable salt, having a particle size distribution such that 90% of the particles are less than 1000 nm, one or more wetting agent and a non-aqueous liquid vehicle, wherein the composition is prepared by a method comprising the steps of—
i. mixing the water soluble, water-susceptible active ingredient or its pharmaceutically acceptable salt, one or more wetting agents, a non-aqueous liquid vehicle,
ii. incorporating at least one inert grinding medium in mixture of step (i),
iii. milling the mixture of step (ii),
iv. separating the at least one inert grinding medium from the milled mixture of step (iii) to obtain a non-aqueous nanosuspension, and
v. converting the non-aqueous nanosuspension of step (iv) into a topical composition.

The method of preparing the nanoparticulate topical composition and/or non-aqueous nanosuspension according to the present invention is described herein in detail with possible alternative steps and process parameters. The water soluble active ingredient or its salt and the wetting agent, can be dispersed or mixed in a non-aqueous liquid vehicle using suitable agitation means such as, for example, stirring, using a roller mill or a cowles type mixer, until a homogeneous dispersion is achieved. Alternatively, the water soluble active ingredient can be dispersed in a premix of liquid vehicle and the wetting agent. This is followed by incorporation of an inert grinding media in mixture and applying mechanical means (milling) to the mixture in the presence of grinding media, so as to reduce the particle size and obtain nanoparticles of the water soluble active ingredient or its salt. The mechanical means used to reduce the effective average particle size of the water soluble active ingredient, conveniently can take the form of dispersion or grinding mill. Suitable dispersion mills include a ball mill, an attrition mill, a vibratory mill, a planetary mill, media mills—such as a sand mill and a bead mill. In preferred embodiments, a media mill is used due to the relatively shorter milling time required to provide the desired reduction in particle size.

The grinding media for the particle size reduction step can be selected from rigid media preferably spherical beads having a mean size less than 3 mm, preferably less than 1 mm, preferably in the range of about 0.07 mm to 1.0 mm, more preferably in the range of about 0.2 mm to 0.4 mm. In one embodiment, a combination of small and large size grinding media may be used. Such media desirably can provide the particles of the invention with shorter processing times and impart less wear to the milling equipment. The selection of the material for the grinding media is believed not to be critical. However, 95% ZrO stabilized with yttrium, magnesia, zirconium silicate, glass, titanium or alumina provide particles having levels of contamination which are believed to be acceptable for the preparation of pharmaceutical compositions. Further, other media, such as glass, stainless steel, titanium, alumina, polymeric beads/resins like crosslinked polystyrene & methyl methacrylate or beads made up of biodegradable polymers, may be used. Preferably, in one embodiment, the grinding media is 95% ZrO stabilized with yttrium

The preferred proportions of the grinding media, the water soluble active agent, the non-aqueous liquid vehicle, and wetting agent present in the grinding vessel can vary within wide limits and depends, for example, upon the size and density of the grinding media, the type of mill selected, etc. The attrition time may vary and depends primarily upon the mechanical means and residence conditions selected, the initial and final particle size and so forth. In one or more embodiments, the milling is carried out for a period of about 30 minutes to about 48 hours. The method can be carried out within a wide range of temperatures and pressures. In preferred embodiments, milling is carried out at a processing temperature of less than 40° C. In preferred embodiments, the processing temperatures of around 20° C. to 40° C. for grinding are ordinarily preferred. If desired, the processing equipment may be cooled with conventional cooling equipment. The method is conveniently carried out under conditions of ambient temperature and at processing pressures which are safe and effective for the milling process and at which the active agent is stable. The grinding media is separated from the milled particulate agent using conventional separation techniques, in a secondary process such as by simple filtration, sieving through a mesh filter or screen, and the like. Other separation techniques such as centrifugation may also be employed to obtain the non-aqueous nanosuspension. In one specific embodiment, milling may be performed by using a bead mill (model—NETZSCH Feinmahltechnik GmbH) comprising beads made up 95% ZrO stabilized with yttrium, having a bead size ranging from about 0.2 mm to 0.4 mm, the milling being carried out at a processing temperature of less than 40° C. and for a period of about 30 minutes or more. According to this embodiment, the nanoparticles of water soluble active ingredient or its pharmaceutically acceptable salts have a particle size distribution such that 90% of the particles (D₉₀) are less than 1000 nm and 50% of the particles (D₅₀) are less than 800 nm.

The non-aqueous nanosuspension so obtained may be converted into a topical composition. This is achieved by mixing the non-aqueous nanosuspension with pharmaceutically acceptable topical non-aqueous liquid vehicle excipients or mixing the pharmaceutically acceptable topical non-aqueous liquid vehicle excipients with the non-aqueous nanosuspension, to obtain the nanoparticulate topical compositions such as gel, foam, lotion or ointment. This can be achieved either by first mixing the excipients of the non-aqueous topical liquid vehicle under appropriate temperature and/or stirring condition to get a excipient mixture with uniform consistency followed by addition of the non-aqueous nanosuspension of the water soluble active ingredient; or alternatively it can be achieved by addition of various topical excipients to the non-aqueous nanosuspension and then mixing under appropriate temperature and/or stirring condition to obtain the topical composition. The sequence and steps of addition of non-aqueous topical vehicle excipients may vary depending upon the dosage form and excipients used.

In one or more embodiments according to the present invention, the nanoparticulate topical composition or the non-aqueous nanosuspension comprises nanoparticles of water soluble, water-susceptible active ingredient or its salt, having a particle size distribution such that 90% of the particles are less than 1000 nm i.e. D₉₀ is less than 1000 nms. In preferred embodiments, the nanoparticles have a particle size distribution such that D₉₀ is less than 1000 nms and (D₅₀) is less than 800 nm. Preferably, the nanoparticles of water soluble active ingredient or its salts have a particle size distribution such that D₉₀ is less than 700 nm, D₅₀ is less than 500 nm, and D₁₀ is less than 300 nms. Suitably, according to the present invention, laser light diffraction technique is preferably used for the determination of particle size and its distribution. The laser light diffraction technique used for the determination of particle size and its distribution is based on the analysis of the diffraction pattern produced when particles are exposed to a beam of monochromatic light. Suitably, the instrument based on this technique that can be preferably used include Malvern Mastersizer or Malvern Zetasizer.

According to the present invention, the nanoparticulate topical composition is suitably a topical dosage form such as a gel, foam, an ointment, a suspension, an aerosol, a spray, a cream or a lotion and the like, which is suitable for topical application. The topical composition is stable, commercially feasible; easy to manufacture and easy to use.

The nanoparticulate topical composition according to one preferred embodiment the present invention is a non-aqueous nanosuspension which may be applied as such or may take the form of a suitable formulation such as spray formulation.

Suitably, the water soluble active ingredient that may be used according to the present invention includes any water soluble active ingredients that are water susceptible. The active agent may be in the form of a pharmaceutically acceptable salt or free base or mixtures thereof. The active ingredient, either in free form or as its salt form, is susceptible to degradation in the presence of water. In one or more embodiment, nanoparticulate topical composition of the present invention includes topically effective water soluble, water-susceptible active ingredients. In certain preferred embodiments, the water soluble, water-susceptible active agent is a tetracycline antibiotic. In one or more embodiments, the tetracycline antibiotic is tetracycline, minocycline, doxycycline, oxytetracycline, demeclocycline, lymecycline, meclocycline, methacycline, rolitetracycline, chlorotetracycline or tigecycline. In certain embodiments the tetracycline is a mixture of two or more tetracyclines. In one or more preferred embodiments the tetracycline is minocycline or its pharmaceutically acceptable salt. The water soluble, water-susceptible active ingredient or its pharmaceutically acceptable salt is present in the nanoparticulate topical composition in therapeutically effective amounts. The concentration of active ingredient will vary with the particular dosage form and the disease state for which it is intended.

Suitably, one or more wetting agents according to some embodiments of the present invention comprise one or more wetting agent having a HLB value from 1 to 10. Preferably the wetting agent is a non-ionic surfactant. More preferably the wetting agent is a non-ionic surfactant having a HLB value from 1 to 10. More preferably, the wetting agent is a non-ionic surfactant which is chemically similar to the non-aqueous liquid vehicle, for example wetting agent is a silicon based surfactant when the non-aqueous liquid vehicle is a silicone fluid. The non-ionic surfactants as the wetting agent that can be used in the context of the present invention includes, but are not limited to Silicon based non-ionic surfactants; Sorbitan esters (ex Span®80); Sucrose stearic acid esters; Glyceryl monostearate, Glyceryl monooleate, Macrogolglycerol; Hydroxy stearates (PEG 7 hydrogenated castor oil), PEGS Castor Oil and the like and mixtures thereof. Non-limiting examples of silicon based non-ionic surfactants that can be used in the context of the present invention includes dimethicone copolyol polymer or cyclomethicone-dimethicone copolyol polymer [(available in market under the brand name DC5225C®, by Dow Corning company). Chemically it is poly(oxyethylene. oxypropylene) methyl polysiloxane copolymer, INCI name is cyclopentasiloxane-PEG/PPG-18/18 Dimethicone)], silicone phosphate ester polymer, a silicone sulfate polymer, a silicone carboxylate polymer, a silicone sulfosuccinate polymer, a silicone sulfonate polymer, a silicone thiosulfate polymer, a silicone amphoteric polymer, a silicone betaine polymer, a silicone phosphobetaine polymer, a silicone alkyl quaternary polymer, a silicone quaternary polymer, a silicone imidazoline quaternary polymer, a silicone carboxy quaternary polymer, a silicone alkanolamide polymer, a silicone ester polymer and mixtures thereof. In preferred embodiments, the nanoparticulate topical composition or nanosuspension comprises the silicon based non-ionic surfactants—cyclomethicone-dimethicone copolyol polymer. Suitably, in preferred embodiments, the nanoparticulate topical composition or nanosuspension is free of ionic surfactants.

Suitably, the concentration of wetting agents used in the method according to the present invention may range from about 0.5% by weight to about 20.0% by weight, such as about 1.0, 1.5, 2.0, 2.5, 3.0, 3.5, 4.0, 4.5, 5.0, 6, 7, 8, 9, 10, 11, 12, 13, 14, 15, 16, 17, 18, or 19% by weight, preferably from about 1.0% by weight to about 10.0% by weight of the composition, more preferably from about 1.0% by weight to about 5.0% by weight of the composition. Suitably, the concentration of wetting agents used in the non-aqueous nanosuspension according to the present invention may range from about 1.0% by weight to about 50.0% by weight, more preferably from about 2.0% by weight to about 40.0% by weight, more preferably from about 3.0% by weight to about 30.0% by weight of the nanosuspension. In preferred embodiments, the ratio of the water soluble active ingredient or its salt to the wetting agent in the non-aqueous nanosuspension can vary from about 1:0.1 to about 1:10.

The nanoparticulate topical composition according to the present invention comprises one or more non-aqueous liquid vehicle. The non-aqueous liquid vehicle excludes aqueous vehicles or protic solvents that contain water, such as for example water, glycols, alcohols, acids or bases. The suitable examples of the non-aqueous vehicle include, but are not limited to, silicon fluids, non-volatile oils or mixtures thereof. It may further include emollients, gelling agents, viscosity builders, or other non-aqueous pharmaceutically acceptable excipients that are suitable for topical application. Suitably, the concentration of non-aqueous liquid vehicle used in the nanoparticulate topical composition and the non-aqueous nanosuspension according to the present invention may range from about 1% to about 99%, from 2.0% w/w to about 95.0% w/w, from about 10.0% w/w to about 95.0% w/w.

In one preferred embodiment, the non-aqueous liquid vehicle comprises a silicon fluid. In another preferred embodiment, the non-aqueous liquid vehicle comprises a mixture of silicon fluid and a non-volatile oil. Suitably, the silicon fluid may be selected from silicones, silicone derivatives or siloxanes. Non limiting example of silicon fluids includes linear or cyclic alkyl siloxanes, aryl siloxanes, alkylether siloxanes, haloalkyl siloxanes, polycycloxanes, siloxane polymers, other functionalized siloxanes and the like and mixtures thereof. In preferred embodiment, the silicon fluid is selected from cyclopoly dimethyl siloxane (cyclomethicone example decamethylcyclopentasiloxane); poly dimethyl siloxane (silicon oils such as dimethicone) or mixture thereof. Other representative silicon fluids that may be used include, hexamethyldisiloxane, decamethyltetrasiloxane, dodecamethylpentasiloxane, tetradecamethylhexasiloxane, hexadecamethylheptasiloxane, hexamethylcyclotrisiloxane, octamethylcyclotetrasiloxane, dodecamethylcyclohexasiloxane. Suitably, the non-volatile oil is selected from mineral oil, paraffin oil, castor oil, olive oil, seasom oil, soybean oil, peanut oil, coconut oil, avocado oil, jojoba oil, grape seed oil, jojaba oil, corn oil, cottonseed oil, white petrolatum, white soft paraffin, shea butter, triglycerides like labrafac, triacetin, capric/caprylic triglyeride, octyl dodecanol, diisopropyl adipate, light mineral oil and the like and mixtures thereof. In a preferred embodiment, the non-aqueous liquid vehicle comprises cyclomethicone or a mixture of cyclomethicone and mineral oil.

In some embodiments, the nanoparticulate topical compositions and/or non-aqueous nanosuspension, according to the present invention may further include excipients such as, but not limited to, a penetration enhancer like isopropyl myristate, isopropyl palmitate, oleic acid etc.; an antioxidant such as butylated hydroxy anisole, butylated hydroxy toluene, tocopherol succinate, propyl gallate, tocopherol, (vitamin E), tocopherol sorbate, tocopherol acetate, other esters of tocopherol, butylated hydroxy benzoic acids and the like; a preservative such as C₁₂ to C₁₅ alkyl benzoates, alkyl p-hydxoxybenzoates, ascorbic acid, benzalkonium chloride, sorbic acid, citric acid, benzoic acid, benzoic acid esters of C₉ to C₁₅ alcohols, chlorocresol, methyl paraben, propyl paraben, sodium benzoate and the like; a surfactant such as a non-ionic surfactant. Other suitable ingredients known in the art, for instance, a tonicity modifier, a viscosity modifier, an anti perspirant, an anti-static agent, a chelating agent, a colorant, a diluent, a humectant, an occlusive agent, a perfuming agent, a sunscreen, or other suitable agents may optionally be incorporated in the topical pharmaceutical compositions of the present invention. Any suitable agent in each group that is non-aqueous and suitable for topical pharmaceutical application may be used. The excipients may be used in suitable amounts known, which can be readily determined by one of ordinary skill in the art, so as to get compositions having desired properties. In one preferred embodiment, the nanoparticulate topical compositions include penetration enhancer like isopropyl myristate, isopropyl palmitate, oleic acid and the like. Suitably, the penetration enhancer may be used in an amount ranging from about 1% to about 30% by weight, preferably from about 5% to 25% by weight, more preferably from about 10% to about 20% by weight.

In preferred embodiments, wherein the nanoparticulate topical composition is a gel, the non-aqueous liquid vehicle comprises a silicon fluid and/or mineral oil, at least one gelling agent and at least one emollient. A penetration enhancer, an antioxidant, a preservative, a viscosity builder such as cetostearyl alcohol and/or a surfactant or other suitable agents may optionally be used.

In one particular embodiment, the non-aqueous nanoparticulate topical composition is a gel and it comprises a water soluble, water-susceptible active ingredient, a wetting agent and a non-aqueous liquid vehicle comprising a silicon fluid, at least one gelling agent, at least one emollient, a viscosity builder such as cetostearyl alcohol, a penetration enhancer and an antioxidant. In another particular embodiment, the non-aqueous nanoparticulate topical composition is a gel and it comprises a water soluble, water-susceptible active ingredient, a wetting agent and a non-aqueous liquid vehicle comprising a silicon fluid, a mineral oil, at least one gelling agent, at least one emollient, a viscosity builder such as cetostearyl alcohol, a penetration enhancer and an antioxidant.

Suitably, the at least one gelling agent that can be used in the nanoparticulate topical gel composition according to the present invention includes, but are not limited to, silicone based gelling/thickening agent such as ‘Elastomer 10®’ which is chemically a crosspolymer of cyclopentasiloxane and dimethicone; ST wax 30®, which is chemically an alkylmethyl silicone wax and the like and mixtures thereof. ST wax 30® also acts as an emollient. Suitably, the at least one emollient that can be used in the topical gel composition according to the present invention includes, but are not limited to, silicone based emollients such as ST wax 30® which is chemically an alkylmethyl silicone wax, Silky wax 30® which is chemically stearoxytrimethylsilane and stearyl alcohol, cyclomethicone, dimethicone, dimethiconol (hydroxy terminated polydimethylsiloxane), disiloxane and the like; other waxes like white ceresin wax (mixture of paraffin and microcrystalline waxes), oily emollients such as mineral oil or other suitable emollients.

Suitably, the at least one emollient that can be used in the topical gel composition according to the present invention includes, but are not limited to, silicone based emollients such as ST wax 30® which is chemically an alkylmethyl silicone wax, Silky wax 30® which is chemically stearoxytrimethylsilane and stearyl alcohol, cyclomethicone, dimethicone, dimethiconol (hydroxy terminated polydimethylsiloxane), disiloxane and the like; other waxes like white ceresin wax (mixture of paraffin and microcrystalline waxes), oily emollients such as mineral oil or other suitable emollients.

In another preferred embodiment, wherein the nanoparticulate topical composition is a foam or an aerosol, the non-aqueous liquid vehicle comprises a silicon fluid and/or mineral oil, at least one foaming agent, at least one surfactant, at least one non-aqueous liquid (that can act as a foam breaking agent), at least one rheology modifier and at least one propellant. A penetration enhancer, an antioxidant, a preservative or other suitable agents used in foam compositions may optionally be used.

In one particular embodiment, the non-aqueous nanoparticulate topical composition is a foam and it comprises a water soluble, water-susceptible active ingredient, a wetting agent and a non-aqueous liquid vehicle comprising a silicon fluid, a mineral oil, at least one foaming agent, at least one surfactant, at least one rheology modifier, at least one non-aqueous liquid which impart foam breakability and at least one propellant.

Suitably, the at least one foaming agent (also known as foam adjuvants) that can be used in the nanoparticulate topical foam composition according to the present invention includes, but are not limited to, oleyl alcohol, stearyl alcohol, myristyl alcohol, cocoglyerides, behenyl alcohol, palmitic acid, stearic acid, oleic acid and the like and mixtures thereof.

Suitably, the at least one propellant that can be used in the foam or aerosol nanoparticulate topical composition according to the present invention, includes, but are not limited to, compressed gases, volatile hydrocarbons such as butane, propane, isobutane, halo hydrocarbon propellants, and the like or mixtures thereof. Preferably, the propellants are hydrocarbon propellants such as NIP-70 (combination of Propane/Isobutane/n-butane in a ratio of 55/15/30 and having a vapor pressure of 70 psig); HARP-AP40 (combination of Propane/Isobutane/n-butane, in a ratio of 22/24/54 and having a vapor pressure of 40 psig) and the like.

Suitably, the at least one non-aqueous liquid that can be used in the foam nanoparticulate topical composition according to the present invention includes silicon fluids and/or oils such as but not limited to disiloxane, cyclomethicone, dimethicone, dimethiconol (hydroxy terminated polydimethylsiloxane), mineral oil and the like and mixtures thereof. These liquids can act as a foam breaking agent or spreading agent.

In another preferred embodiment, wherein the nanoparticulate topical composition is an ointment or a lotion, the non-aqueous liquid vehicle comprises a silicon fluid and/or mineral oil, at least one non-aqueous liquid (which acts as a spreading agent), at least one rheology modifier, at least one surfactant, at least one ointment base like petrolatum. A penetration enhancer, an antioxidant, a preservative or other suitable agents used in formulating ointment/lotion compositions, may optionally be used.

The at least one surfactant that can be used in the gel, foam, aerosol, ointment, lotion composition according to the present invention, preferably includes a non-ionic surfactant such as silicon based non-ionic surfactants such as dimethicone copolyol polymer or cyclomethicone-dimethicone copolyol polymer; sorbitan esters such as Span®80; sucrose stearic acid esters; glyceryl monostearate, glyceryl monooleate, macrogolglycerol; hydroxy stearates (PEG 7 hydrogenated castor oil), PEGS castor oil and the like and mixtures thereof.

Suitably, the at least one rheology modifier that can be used in the foam or aerosol or ointment or lotion type nanoparticulate topical composition according to the present invention, includes, but are not limited to, silicone based thickening agent such as ‘Elastomer 10®’ (crosspolymer of cyclopentasiloxane and dimethicone); ST wax 30®; Gelucire®43/01 (glycerol esters of saturated C₁₂-C₁₈ fatty acids); petrolatum, or other suitable agents and mixtures thereof.

According to one particularly preferred embodiment, the method of the present invention provides a topical composition of minocycline or its pharmaceutically acceptable salt. Preferably, minocycline or its pharmaceutically acceptable salts is Minocycline hydrochloride, which has the following structure:

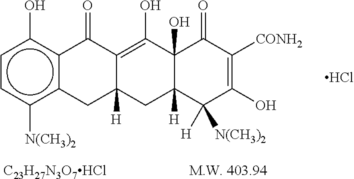

Minocycline or its pharmaceutically acceptable salt is present in the compositions in therapeutically effective amounts. Preferably, the effective amount of Minocycline or its pharmaceutically acceptable salt present in the nanoparticulate topical composition is such that it is sufficient to treat or prevent acne, rosacea or related disorders of the skin when applied topically. The dosages of minocycline salts will be understood to be on the basis of the amount of minocycline free base provided thereby, and thus may be expressed as a minocycline free base equivalent dosage or amount. Minocycline or its pharmaceutically acceptable salt is present in the non-aqueous nanosuspension at a concentration ranging from about 0.01% to about 15% by weight, such as about 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.74, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14 or 15% by weight, preferably about 0.1% to about 10% by weight, more preferably about 0.5% to about 5% by weight of the nanosuspension. The nanoparticulate topical composition typically contain an effective amount, e.g., about 0.01% to about 10% by weight (w/w), such as about 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.74, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5 or 10% by weight, preferably about 0.01% to about 5% by weight, more preferably about 0.1% to about 3% by weight, more preferably about 0.2% to about 1.5% by weight of minocycline or its salt. The concentration of active ingredient will vary with the particular dosage form and the disease state for which it is intended. In most preferred embodiments, the active agent is present in the nanoparticulate topical pharmaceutical composition at a concentration of about 0.5% or 1.0% by weight. In one preferred embodiment, minocycline hydrochloride used in the nanoparticulate composition of the present invention is crystalline in nature. In one embodiment, the crystalline nature of the active is maintained even after nano-milling and the non-aqueous nanosuspension and the topical composition of the present invention essentially comprises minocycline in crystalline form.

In one specific embodiment, the present invention provides a method of preparing a nanoparticulate minocycline topical composition, said method comprising mixing minocycline or its pharmaceutically acceptable salts with one or more wetting agents and a non-aqueous liquid vehicle, adding inert grinding media to the above mixture; milling the mixture; and separating the inert grinding media to obtain a non-aqueous nanosuspension of minocycline or its salt and converting the non-aqueous nanosuspension into a nanoparticulate minocycline topical composition by mixing the non-aqueous nanosuspension with pharmaceutically acceptable topical vehicle excipients.

In one specific embodiment, the present invention provides a nanoparticulate minocycline topical composition, comprising minocycline or its pharmaceutically acceptable salts, one or more wetting agents and a non-aqueous liquid vehicle, prepared according to the method hereinabove described.

In one embodiment, the present invention provides a nanoparticulate minocycline topical composition comprising minocycline or its pharmaceutically acceptable salt having a particle size distribution such that 90% of the particles (D₉₀) are less than 1000 nm in diameter, one or more wetting agents and a non-aqueous liquid vehicle, prepared by a method comprising steps of mixing minocycline or its pharmaceutically acceptable salts, one or more wetting agents and a non-aqueous liquid vehicle; incorporating inert grinding media to the above mixture; milling the mixture; and separating the inert grinding media to obtain a non-aqueous nanosuspension, and converting the non-aqueous nanosuspension into a nanoparticulate minocycline topical composition by mixing the non-aqueous nanosuspension with pharmaceutically acceptable topical vehicle excipients.

In one specific embodiment according to the present invention, the nanoparticulate topical composition is a gel, comprising nanoparticulate minocycline or its pharmaceutically acceptable salts, one or more wetting agents, a non-aqueous liquid vehicle, including pharmaceutically acceptable topical gel vehicle excipients. In another specific embodiment according to the present invention, the nanoparticulate topical composition is a foam, comprising nanoparticulate minocycline or its pharmaceutically acceptable salts, one or more wetting agents, a non-aqueous liquid vehicle including pharmaceutically acceptable topical foam vehicle excipients.

According to preferred embodiments of the invention, the particle size of minocycline or its pharmaceutically acceptable salt present in the nanoparticulate minocycline topical composition is such that (D₅₀) is less than 800 nm and D₉₀ is less than 1000 nms. According to preferred embodiments of the invention, the one or more wetting agent is cyclomethicone-dimethicone copolyol polymer (a silicon based non-ionic surfactants) and the non-aqueous liquid vehicle comprises cyclomethicone or a mixture of cyclomethicone and mineral oil. It may further comprise other pharmaceutically acceptable topical non-aqueous liquid vehicle excipients.

In a particularly preferred embodiment, the present invention provides a method of preparing a nanoparticulate topical composition of minocycline or its pharmaceutically acceptable salt, said method comprising steps of—
i. mixing minocycline or its pharmaceutically acceptable salt, one or more wetting agents, a non-aqueous liquid vehicle
ii. incorporating at least one inert grinding medium in mixture of step (i),
iii. milling the mixture of step (ii)
iv. separating the at least one inert grinding medium from the milled mixture of step (iii) to obtain a non-aqueous nanosuspension, and
v. converting the non-aqueous nanosuspension of step (iv) into a topical composition wherein the method does not involve use of water, and wherein the topical composition comprise minocycline or its pharmaceutically acceptable salt, having a particle size distribution such that 90% of the particles (D₉₀) are less than 1000 nm, and 50% of the particles (D₅₀) are less than 800 nm, wherein the wetting agent is cyclomethicone-dimethicone copolyol polymer and the non-aqueous liquid vehicle comprises cyclomethicone or mineral oil or mixture thereof.

The nanoparticulate topical compositions according to the present invention such as the non-aqueous nanosuspensions, the gel and foam compositions were found to be physically and chemically stable upon manufacture and storage. The non-aqueous nanosuspension of the present invention show proper suspension behavior and is physically stable for at least three months. No significant change in particle size distribution of minocycline or its salt was observed upon storage. Further, the nanosuspension as well as nanoparticulate topical compositions did not showed any sign of chemical degradation. The chemical assay of minocycline did not substantially change upon storage and remains within the specified limit of 90-110% of label claim. The impurity profile or contents of related substances or total impurities remains within the specified limits, of not more than 4% upon storage.

The nanoparticulate topical compositions of the present invention are useful in the treatment of acne, rosacea, impetigo or a skin disease caused by bacteria (such as Staphylococcus aureus, Streptococcus pyogenes, Escherichia coli, Pseudomonas aeruginosa, a methicillin resistant Staphylococcus aureus bacteria), by topical application of the nanoparticulate topical compositions to the affected diseased area of the skin, mucosa or eye. The present invention provides a method of treating acne, rosacea, impetigo or a skin disease caused by bacteria, by topical application of a non-aqueous nanoparticulate topical composition comprising nanoparticles of a water soluble active ingredient or its pharmaceutically acceptable salt, having a particle size distribution such that 90% of the particles are less than 1000 nm. The present inventors have discovered that compositions containing nanoparticles of minocycline or its pharmaceutically acceptable salts, according to the present invention provides improved efficacy in treating acne.

In one embodiment there is provided a nanoparticulate topical composition manufactured according to the method of the present disclosure, for use as a medicament.

In one embodiment there is provided a nanoparticulate topical composition manufactured according to the method of the present disclosure, for use in the treatment of acne, rosacea, impetigo or a skin disease caused by bacteria.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

Hereinafter, the invention will be more specifically described by way of Examples. The examples are not intended to limit the scope of the invention and are merely used as illustrations.

### EXAMPLES

Examples 1-5 gives the composition and process of preparing the topical non-aqueous nanosuspension composition of minocycline hydrochloride.

TABLE 1
Details of the Non-aqueous nanosuspension composition

| Category | | Quantity (in grams) | | | | |
|---|---|---|---|---|---|---|
| of Ingre- | | Exam- | Exam- | Exam- | Exam- | Exam- |
| dients | Ingredient | ple 1 | ple 2 | ple 3 | ple 4 | ple 5 |
| Active | Minocycline | 5.6 | 3.07 | 3.00 | 3.07 | 7.31 |
| agent | Hydro- | | | | | |
| | chloride | | | | | |
| Non- | Cyclo- | 140.0 | 76.86 | 39.0 | 76.86 | 91.4 |
| aqueous | methicone | | | | | |
| Liquid | | | | | | |
| vehicle | | | | | | |
| Non- | Mineral | — | — | 36.0 | 36.0 | — |
| aqueous | Oil | | | | | |
| Liquid | | | | | | |
| vehicle | | | | | | |
| Wetting | Cyclo- | 6.0 | 7.07 | 9.00 | 6.9 | 14.62 |
| agent | methicone | | | | | |
| | Dimethicone | | | | | |
| | Copolyol | | | | | |

Method of preparation of non-aqueous nanosuspension: Minocycline was dispersed in cyclomethicone along with cyclomethicone dimethicone copolyol and mixed. To this was added, inert grinding media made up of 95% ZrO stabilized with magnesia and having diameter of 0.4 mm. The mixture was stirred for about 24 hours and milling carried out. The inert grinding media was separated and the resulting nanosuspension was analysed for recording the ‘particle size distribution’ of minocycline nanoparticles using Malvern Mastersizer (MS3000).

The results of Malvern particle size analysis for nanosuspensions of example 1-5 is presented below in Table 2.

TABLE 2
Particle Size Distribution (PSD) Results
for nanosuspension of Example 1-5:

| PSD | | | | | |
|---|---|---|---|---|---|
| (nm) | Example 1 | Example 2 | Example 3 | Example 4 | Example 5 |
| D₁₀ | 235 | 177 | 250 | 146 | 187 |
| D₅₀ | 368 | 256 | 393 | 232 | 274 |
| D₉₀ | 589 | 369 | 613 | 339 | 403 |

In various examples, the mean particle size of the minocycline hydrochloride is such that 50% of the particles (D₅₀) have a diameter varying from 200 nms to about 400 nms, and 90% of the particles (D₉₀) have a diameter of less than 1000 nms.

The non-aqueous nanosuspension of Example 1 & 5 were subjected to storage stability study by keeping the nanosuspension in an amber colored glass vial at room temperature (25° C./60% relative humidity) for at least 3 months. The physical appearance, change in particle size distribution, and chemical assay of Minocycline hydrochloride were evaluated after 3 months. The analysis of assay of minocycline hydrochloride, related substances and total impurities was done using HPLC technique. The observations are given in Table 3 & 4 below:

TABLE 3
Stability study results of nano-suspension of Example 1:

| | Particle Size | | | | |
|---|---|---|---|---|---|
| | Distribution (nm) | | | Chemical | |
| Time point | D₁₀ | D₅₀ | D₉₀ | Assay | Physical Appearance |
| Initial | 235 | 368 | 589 | 99.43% | Suspension |
| 3 Month | 210 | 311 | 463 | 104.73% | Suspension |

TABLE 4
Stability study results of nano-suspension of Example 5:

| | Particle Size | | | | | |
|---|---|---|---|---|---|---|
| Time | Distribution (nm) | | | Chemical | 4-Epi | Physical |
| point | D₁₀ | D₅₀ | D₉₀ | Assay | Minocycline | Appearance |
| Initial | 187 | 274 | 403 | 107.24 | 0.89 | Suspension |
| 3 Month | 188 | 273 | 400 | 105.65 | 0.91 | Suspension |

The non-aqueous nanosuspension of the present invention was found to be physically and chemically stable upon manufacture and storage for at least 3 months. No significant change in particle size distribution of minocycline or its salt was observed upon storage. Further, the nanosuspension did not showed any sign of chemical degradation as the chemical assay of minocycline did not changed upon storage. The contents of related substances and total impurities remained within the specified limits, upon storage.

### Examples 6-10

Following nanoparticulate topical composition of minocycline hydrochloride were prepared according to the method of the present invention. The non-aqueous nanosupension (prepared as per method described in Examples 1-5) were converted into topical compositions in the form of a gel whose details are given below in Table 5:

TABLE 5
Details of the nanoparticulate topical composition in the form of gel:

| | | Function of | Amount- % w/w | | | | |
|---|---|---|---|---|---|---|---|
| Description | Ingredients | Ingredients | Example 6 | Example 7 | Example 8 | Example 9 | Example 10 |
| ** Non- | Minocycline | Active agent | 1.0 | 1.0 | 1.0 | 1.0 | 1.0 |
| aqueous | hydrochloride | | | | | | |
| nanosus- | Cyclomethicone | Non aqueous | 25.0 | 13.0 | 25.0 | 25.0 | 25.0 |
| pension | | Vehicle | | | | | |
| | Cyclomethicone | Wetting agent | 2.3 | 2.3 | 2.3 | 2.3 | 2.3 |
| | Dimethicone Copolyol | | | | | | |
| | Mineral Oil | Non aqueous | — | 12.0 | — | — | — |
| | | Vehicle | | | | | |
| Topical | Alkylmethyl silicone | Emollient and | 4.0 | 4.0 | 4.0 | 4.0 | — |
| gel non- | wax (ST Wax 30) | thickener | | | | | |
| aqueous | White Ceresin Wax | Emollient and | — | — | — | — | 4.0 |
| liquid | | thickener | | | | | |
| vehicle | Oleic acid | Penetration enhancer | — | — | 10.0 | — | — |
| | Isopropyl myristate | Penetration enhancer | — | — | — | 20.0 | 20 |
| | Cetostearyl Alcohol | Viscosity builder | 8.0 | 8.0 | 8.0 | 8.0 | 8.0 |
| | Butylated Hydroxy | Antioxidant | 0.1 | 0.1 | 0.1 | 0.1 | 0.1 |
| | Anisole | | | | | | |
| | Cyclopentasiloxane | Gelling agent | q.s. to 100 | q.s. to 100 | q.s to 100 | q.s to 100 | q.s to 100 |
| | and dimethicone | | | | | | |
| | crosspolymer | | | | | | |
| | (Elastomer 10) | | | | | | |

** The non-aqueous nanosuspensions used in formulating the gel compositions comprised of nanoparticles of minocycline having a particle size distribution such that 90% of the particles (D₉₀) have a size in the range of 200 to 700 nms, 50% of the particles (D₅₀) have a size in the range of 100 to 500 nms, and 10% of the particles (D₁₀) have a size in the range of 50 to 300 nms.

Method of preparation of nanoparticulate minocycline topical gel composition: ST wax 30 and cetostearyl alcohol were melted at a temperature of 70-75° C. and butylated hydroxy anisole (and oleic acid as in example 8 or isopropyl myristate as in example 9 & 10) was added to this mixture. The melted mixture was added to Elastomer10 under stirring. To this was added minocycline hydrochloride nanosuspension (containing minocycline hydrochloride, cyclomethicone and/or mineral oil, and cyclomethicone dimethicone copolyol, prepared as per method of example 1-5) and the mixture was stirred at 35° C. to attain uniform consistency. This resulted in the formation of a non-greasy, anhydrous topical gel composition.

The non-aqueous nanoparticulate compositions so prepared were subjected to storage stability testing by storing the composition at room temperature (25° C./60% relative humidity) in white collapsible tube for at least 3 months. The physical appearance, change in particle size distribution, and chemical assay of minocycline hydrochloride were evaluated after 3 months. It was observed that the compositions were physically and chemically stable upon manufacture and storage for at least 3 months. There occurred no change in physical appearance of the compositions (light yellow coloured semisolid gel) upon storage. The viscosity of the composition also did not change substantially upon storage. Further, the nanosuspension did not showed any sign of chemical degradation as the chemical assay of minocycline was well within the limit of 90%-110% of the label claim upon storage. The related substances and total impurities remained within the specified limits of not more than 4%, upon storage. The observations for composition of Example 10 are given in Table 6 below:

TABLE 6
Stability results of nanoparticulate composition:

| | Impurity: | | | |
|---|---|---|---|---|
| Time | 4-Epi- | Assay of | Physical | Viscosity |
| point | minocycline | Minocycline | Appearance | (cps) |
| Initial | 0.923 | 105.32 | Light yellow semi- | 250333 |
| | | | solid gel | |
| 3 Month | 0.967 | 107.04 | Light yellow semi- | 251944 |
| | | | solid gel | |

Note:
The analysis of assay of minocycline hydrochloride and 4-epiminocycline was done using HPLC technique. The viscosity of the composition was determined using a Brookfield ® LVDP + Pro II viscometer at a temperature of 30 ± 2° C.

### Example 11-12

Example 11 and 12 provide the details of the nanoparticulate topical foam composition prepared according to the method of the present invention. The non-aqueous nanosupension (prepared as per the method described in Example 1-5) were converted into topical compositions in the form of foam, whose details are given below in Table 7:

TABLE 7
Details of the topical composition in the form of foam:

| | | Function of | Amount - % w/w | |
|---|---|---|---|---|
| Description | Ingredients | Ingredients | Example 11 | Example 12 |
| **Non-aqueous | Minocycline | Active agent | 1.0 | 1.0 |
| nanosuspension | hydrochloride | | | |
| | Cyclomethicone | Non aqueous Vehicle | 25.0 | 13.0 |
| | Cyclomethicone | Wetting agent | 2.3 | 2.3 |
| | Dimethicone Copolyol | | | |
| | Mineral Oil | Non aqueous Vehicle | — | 12.0 |
| Topical foam | Stearyl alcohol | Foam adjuvants | 2.0 | 2.0 |
| non-aqueous | Cetyl alcohol | Foam adjuvants | 1.0 | 1.0 |
| liquid vehicle | Glyceryl monosteaate | Non-ionic surfactant | 6.0 | 6.0 |
| | Glycerol ester of higher | Hard fat | 6.0 | 6.0 |
| | saturated fatty acid | | | |
| | (Gelucire 43/01) | | | |
| | Mineral oil | Oil phase | 20.0 | 12.0 |
| | Disiloxane (hexadimethyl | Impart Foam | 26.7 | 34.7 |
| | disiloxane and | breakability | | |
| | octamethyltrisiloxane) | | | |
| | Cyclopentasiloxane and | Rheology modifier | 10.0 | 10.0 |
| | dimethicone crosspolymer | | | |
| | (Elastomer 10) | | | |

| | | | |
|---|---|---|---|
| Propellant | Hydrocarbon propellant - | Foam Propellant | 4-20% of foam composition |
| | propane/isobutane/n- | | |
| | butane, 55/15/30 (NIP-70) | | |

**The non aqueous nanosuspensions used in formulating the gel compositions comprised of nanoparticles of minocycline having a particle size distribution such that 90% of the particles (D₉₀) have a size in the range of 200 to 700 nms, 50% of the particles (D₅₀) have a size in the range of 100 to 500 nms, and 10% of the particles (D₁₀) have a size in the range of 50 to 300 nms.

Preparation of nanoparticulate minocycline topical foam composition: The excipients of foam composition vehicle including Stearyl alcohol, Cetyl alcohol, Glyceryl monosteaate, Gelucire, mineral oil, and Elastomer 10 (except disiloxane) were melted at a temperature of 70° C.-75° C. under stirring to attain a mixture with uniform consistency. The mixture was then cooled to 35° C. and to this, the minocycline hydrochloride nanosuspension (containing minocycline hydrochloride, cyclomethicone and/or mineral oil, and cyclomethicone dimethicone copolyol, prepared as per example 1-4) was added along with disiloxane. The dispersion so obtained had a viscosity of about 3720 cps (as determined by a Brookfield® LVDP+Pro II viscometer at a temperature of 25±2° C.). The dispersion was filled in the foam canister and sealed followed by addition of appropriate amount of propellant. This resulted in the formation of a creamy, quick breaking nanoparticulate topical foam composition.

1. A method of preparing a nanoparticulate topical composition, said method comprising the steps of
i. mixing a water soluble, water-susceptible active ingredient or its pharmaceutically acceptable salt, one or more wetting agents and a non-aqueous liquid vehicle
ii. incorporating at least one inert grinding medium in mixture of step (i),
iii. milling the mixture of step (ii),
iv. separating the at least one inert grinding medium from the milled mixture of step (iii), to obtain a non-aqueous nanosuspension, and
v. converting the non-aqueous nanosuspension of step (iv) into a topical composition wherein the method does not involve use of water.

2. The method as claimed in claim 1, wherein the nanoparticulate topical composition comprises nanoparticles of water soluble, water-susceptible active ingredient or its pharmaceutically acceptable salts, having a particle size distribution such that 90% of the particles (D₉₀) are less than 1000 nm and 50% of the particles (D₅₀) are less than 800 nm in size.

3. The method as claimed in claim 1, wherein the nanoparticulate topical composition comprises nanoparticles of water soluble, water-susceptible active ingredient or its pharmaceutically acceptable salts, having a particle size distribution such that 90% of the particles (D₉₀) are less than 700 nm, 50% of the particles (D₅₀) are less than 500 nm and 10% of the particles (D₁₀) are less than 300 nm in size.

4. The method as claimed in claim 1, wherein water soluble, water-susceptible active ingredient is minocycline or its pharmaceutically acceptable salt.

5. The method as claimed in claim 1, wherein the at least one inert grinding medium comprises beads made up of 95% ZrO stabilized with yttrium.

6. The method as claimed in claim 1, wherein the at least one inert grinding medium comprises beads having size ranging from about 0.07 mm to about 1.0 mm.

7. The method as claimed in claim 1, wherein milling is carried out at a processing temperature of less than 40° C.

8. The method as claimed in claim 1, wherein milling is carried out for a period of about 30 minutes to about 48 hours.

9. The method as claimed in claim 1, wherein milling is performed by using a bead mill comprising beads made up 95% ZrO stabilized with yttrium, having a bead size ranging from about 0.2 mm to 0.4 mm, the milling being carried out at a processing temperature of less than 40° C. and for a period of about 30 minutes or more, and wherein the resulting nanoparticles of water soluble active ingredient or its pharmaceutically acceptable salts have a particle size distribution such that 90% of the particles (D₉₀) are less than 1000 nm and 50% of the particles (D₅₀) are less than 800 nm.

10. The method as claimed in claim 4, wherein the water soluble active ingredient is minocycline hydrochloride and it is present at a concentration ranging from about 0.01% to about 15% by weight.

11. The method as claimed in claim 1, wherein the wetting agent is a non-ionic surfactant.

12. The method as claimed in claim 11, wherein the wetting agent is a silicon based non-ionic surfactants such as cyclomethicone-dimethicone copolyol polymer.

13. The method as claimed in claim 1, wherein the wetting agent is present at a concentration ranging from about 0.5% to 20.0% by weight of the composition.

14. The method as claimed in claim 1, wherein the non-aqueous liquid vehicle comprises a silicone fluid or an oil or mixtures thereof, wherein the silicon fluid is selected from the group consisting of linear or cyclic alkyl siloxanes, aryl siloxanes, alkylether siloxanes, haloalkyl siloxanes, polycyclosiloxanes, siloxane polymers, functionalized siloxanes, or mixtures thereof and the oil is selected from mineral oil, paraffin oil, castor oil, olive oil, seasom oil, soybean oil, peanut oil, coconut oil, avocado oil, jojoba oil, grape seed oil, jojaba oil, corn oil, cottonseed oil, white petrolatum, white soft paraffin, shea butter, triglycerides like labrafac, triacetin, capric/caprylic triglyeride, octyl dodecanol, diisopropyl adipate, light mineral oil or mixtures thereof.

15. The method as claimed in claim 1, wherein the topical composition is a gel, a foam, an ointment, a suspension, an aerosol, a spray, a cream or a lotion.

16. A method of preparing a nanoparticulate topical composition, said method comprising steps of—
i. mixing minocycline or its pharmaceutically acceptable salt, one or more wetting agents, a non-aqueous liquid vehicle
ii. incorporating at least one inert grinding medium in mixture of step (i),
iii. milling the mixture of step (ii),
iv. separating the at least one inert grinding medium from the milled mixture of step (iii), to obtain a non-aqueous nanosuspension, and
v. converting the non-aqueous nanosuspension of step (iv) into a topical composition,
wherein the method does not involve use of water and wherein the topical composition comprise minocycline or its pharmaceutically acceptable salt, having a particle size distribution such that 90% of the particles (D₉₀) are less than 1000 nm, 50% of the particles (D₅₀) are less than 800 nm, and the wetting agent is cyclomethicone-dimethicone copolyol polymer and the non-aqueous liquid vehicle comprise cyclomethicone or mineral oil or mixture thereof.

17. A nanoparticulate topical composition comprising nanoparticles of a water soluble, water-susceptible active ingredient or its pharmaceutically acceptable salt, having a particle size distribution such that 90% of the particles are less than 1000 nm, one or more wetting agent and a non-aqueous liquid vehicle, wherein the composition is prepared by a method comprising the steps of—
i. mixing the water soluble, water-susceptible active ingredient or its pharmaceutically acceptable salt, one or more wetting agents, a non-aqueous liquid vehicle
ii. incorporating at least one inert grinding medium in mixture of step (i),
iii. milling the mixture of step (ii),
iv. separating the at least one inert grinding medium from the milled mixture of step (iii), to obtain a non-aqueous nanosuspension, and
v. converting the non-aqueous nanosuspension of step (iv) into a topical composition.
